(19) United States
(12) Patent Application Publication
(10) Pub. No.: US 2007/0224115 A1
(43) Pub. Date: Sep. 27, 2007

(54) Polymeric chelators for radionuclide delivery systems

(75) Inventors: Mary E. Barr, Los Alamos, NM (US); Robert W. Atcher, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: The Regents of the University of California

(21) Appl. No.: 11/388,177

(22) Filed: Mar. 22, 2006

(51) Int. Cl. A61K 51/00

(52) U.S. Cl. 424/1.11; 424/1.49; 424/1.69

(57) ABSTRACT

A radionuclide delivery system that is based upon amorphous, water-soluble polymeric chelators. Each water-soluble polymeric chelator is capable of delivering hundreds of radionuclide atoms to the target. The chemical and physical characteristics of the globular polymer increase the effective decay-particle yield at the target cells and reduce ancillary toxicity due to errant daughter radionuclides by retaining or rebinding daughter nuclei. The delivery system includes a water-soluble polymeric chelator having a plurality of chelating groups; and a plurality of radionuclide atoms bound to a least a portion of the chelating groups, wherein the plurality of chelating groups immobilize at least a portion of the plurality of radionuclide atoms.

### STATEMENT REGARDING FEDERAL RIGHTS

This invention was made with government support under Contract No. W-7405-ENG-36, awarded by the U.S. Department of Energy. The government has certain rights in the invention.

### BACKGROUND OF INVENTION

The invention relates to a system for delivery of radionuclide atoms to a target. More particularly, the invention relates to a system for delivery of radionuclide atoms to a biological target.

Radioactive isotopes that emit particulate radiation, such as radionuclides that are high linear energy transfer (LET) alpha or low LET beta particle emitters, have been used for radiotherapeutic applications. Advantages provided by particle emitters with high LET include the lack of an oxygen effect, cytotoxicity that is independent of dose rate, and the potential to minimize non-target dose if the high LET-containing conjugate stays intact and if targeting takes place quickly and efficiently.

Delivery systems for radionuclides typically have two components: a chelator that binds a metal radionuclide; and a cellular recognition agent that attaches to targeted cells. Such systems currently use chelators that are conjugated to targeting molecules in ratios of one-to-one or several-to-one, with each chelator potentially binding one radionuclide atom. Thus, these delivery systems have a low capacity, as they are only able to accommodate a single radionuclide atom or, at best, a few (i.e, less than about 10) such atoms.

Alpha particles are emitted with an energy of 5-8 MeV. Their large mass (4 amu) and high energy create a dense ionization track which, in turn, deposits that energy in a short path length into the surrounding tissue, causing significant cell damage within a very limited area. Because they combine high cytotoxicity with an irradiation range of only a few cells, heavy element alpha-emitters show promise for the treatment of disseminated diseased tissue.

In the case of alpha-emitters, two challenges have been the limited availability of many of the radionuclides suitable for use in therapy and their relatively short half-lives. In some instances, the therapeutic isotope is not itself an alpha-emitter, but is instead a conveniently long-lived precursor to an extremely short-lived alpha-emitting daughter. In other cases the alpha-emitting parents are sufficiently long-lived for therapeutic applications.

Daughter nuclei of alpha-emitters generally have a recoil energy of about 100 keV. This energy is sufficient to break all chemical bonds to the daughter nucleus and cause substantial damage to the chelate construct. Although the linear penetration range for recoil nuclei is relatively short (<100 nm), half-lives on the order of a few seconds could allow for significant migration of daughters away from the target sites. In addition, radioactive daughter migration from the active site may cause ancillary toxicity in some cases. For example, ancillary toxicity is a serious issue for the Ac-225 system. Furthermore, uncertainties related to daughter nuclide diffusion complicate dosimetric analysis and predictive capability.

Current radionuclide delivery systems are limited in their capacity to deliver an effective dose of radionuclides to a biological target. Furthermore, such systems generally do not effectively limit daughter migration. Therefore, what is needed is a delivery system that can accommodate multiple radionuclides. What is also needed is a delivery system that is capable of limiting daughter migration away from target sites.

### SUMMARY OF INVENTION

The present invention meets these and other needs by providing a radionuclide delivery system that is based upon amorphous, water-soluble polymeric chelators. Each water-soluble polymeric chelator is capable of delivering up to hundreds of radionuclide atoms to the target. The chemical and physical characteristics of the globular polymer increase the effective decay-particle yield at the target cells and reduce ancillary toxicity due to errant daughter radionuclides by retaining or rebinding daughter nuclei.

Accordingly, one aspect of the invention is to provide a radionuclide delivery system. The delivery system comprises: a water-soluble polymeric chelator having a plurality of chelating groups; and a plurality of radionuclide atoms, wherein each radionuclide atom is bound to one of the chelating groups, and wherein the plurality of chelating groups immobilize at least a portion of the plurality of radionuclide atoms.

A second aspect of the invention is to provide a water-soluble polymeric chelator. The water-soluble polymeric chelator is a polyethyleneimine derivative having a plurality of chelating groups, wherein each of the chelating groups is capable of binding and immobilizing a radionuclide atom.

A third aspect of the invention is to provide a radionuclide delivery system. The delivery system comprises: a water-soluble polymeric chelator having a plurality of chelating groups, wherein each of the chelating groups is capable of binding and immobilizing a radionuclide atom; a plurality of radionuclide atoms, wherein each radionuclide atom is bound to one of the chelating groups; and a target moiety bound to the water-soluble polymeric chelator.

A fourth aspect of the invention is to provide a method of making a radionuclide delivery system comprising a water-soluble polymeric chelator having a plurality of chelating groups, wherein each of the chelating groups is capable of binding and immobilizing a radionuclide atom, and a plurality of radionuclide atoms, wherein each radionuclide atom is bound to one of the chelating groups. The method comprises the steps of: providing the water-soluble polymeric chelator; providing the plurality of chelating groups; providing the plurality of radionuclide atoms; and forming a water-soluble polymeric chelator-radionuclide construct, wherein a portion of the radionuclide atoms bind to a portion of the chelator groups and the chelator groups are linked to the water-soluble polymeric chelator.

These and other aspects, advantages, and salient features of the present invention will become apparent from the following detailed description, the accompanying drawings, and the appended claims.

### BRIEF DESCRIPTION OF THE DRAWINGS

FIG. 1 is a schematic representation of a radionuclide delivery system;

FIG. 2 shows structures of: a) a cross-linked parent polyethyleneimine (PEI) polymer; b) carboxylated PEI; and c) PEI with phosphoric acids groups;

FIG. 3 is a table containing metal-polymer binding data for the radionuclides (with error limits in parentheses), media, and polymers studied;

FIG. 4 is a table containing binding data of Am-241 to polyethyleneimine derivatized with diphosphonic acids (PEIDP) in the presence of Na, Ca, and Fe ions; and

FIG. 5 is a flow chart for a method of making a radionuclide delivery system.

### DETAILED DESCRIPTION

In the following description, like reference characters designate like or corresponding parts throughout the several views shown in the figures. It is also understood that terms such as “top,” “bottom,” “outward,” “inward,” and the like are words of convenience and are not to be construed as limiting terms. In addition, whenever a group is described as either comprising or consisting of at least one of a group of elements and combinations thereof, it is understood that the group may comprise or consist of any number of those elements recited, either individually or in combination with each other.

The invention provides a radionuclide delivery system based upon amorphous water-soluble polymeric chelators. Whereas typical targeting molecule-chelator systems bind a single radionuclide atom, the radionuclide delivery system described herein is capable of binding hundreds of radionuclide atoms. These systems may increase and modulate the effective dose at target cells by providing high radionuclide capacity. The system is a dose-tunable system: it may be used to be adjusted in real time to acquire activity loading levels that are appropriate for the individual therapy. Alternatively, the large excess of active-metal binding sites allow the use of isotopically dilute media, thus reducing the need for chemical or isotopic separations of reactor targets.

Another feature of the invention is applicable to multiple alpha-emitting radionuclides. The globular polymer's chemical and physical characteristics increase the effective decay-particle yield at the target cells while reducing ancillary toxicity due to errant daughter radionuclides by retaining or rebinding the daughter nuclei.

The invention includes a system having applications in diagnostic and imaging as well as in high-dose cancer treatment. For in vivo use, the polymeric chelators may be attached to cell-specific targeting molecules such as proteins, monoclonal antibodies, and the like. Alternatively, the delivery system may be injected directly at localized tumors, or as a colloid.

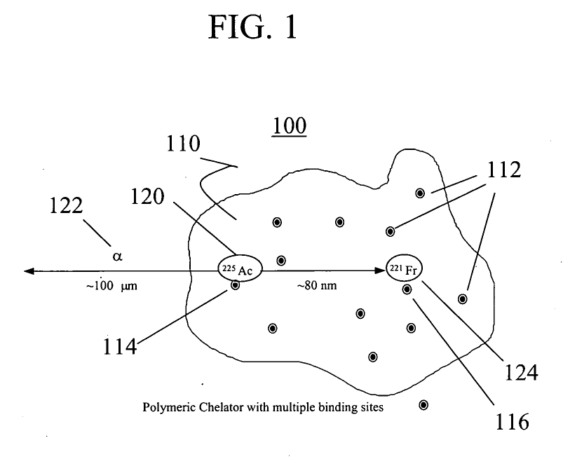

Referring to the drawings in general and to FIG. 1 in particular, it will be understood that the illustrations are for the purpose of describing a particular embodiment of the invention and are not intended to limit the invention thereto. A schematic representation of the radionuclide delivery system is shown in FIG. 1. Radionuclide delivery system 100 includes a water-soluble polymeric chelator 110. Water-soluble polymeric chelator 110 includes a plurality of chelating groups, which act as binding sites 112 for radionuclide atom 120. Binding sites 112 serve to immobilize radionuclide atoms 120 as well as daughter atoms of radionuclide atoms 122 that are created by radioactive decay of radionuclide atoms 120.

In FIG. 1, Ac-225 is depicted as the radionuclide atom 120, located at binding site 114. Radionuclide atom 120 emits an alpha particle 122 and daughter nucleus 124 is ejected. Fr-221, the daughter of Ac-225, is shown in FIG. 1. Daughter nucleus 124 travels through a portion of water-soluble polymeric chelator 110. Rather than escaping from water-soluble polymeric chelator 110, daughter nucleus 124 is captured at a second binding site 116. While the radionuclide Ac-225 and daughter nucleus Fr-221 are shown in FIG. 1, it is understood that other radionuclides and their respective daughter nuclei may be bound at binding sites 112, 114, 116.

In one embodiment, radionuclide delivery system 100 further comprises a targeting moiety (not shown) attached to water-soluble polymeric chelator 110. Possible targeting moieties include, but are not limited to, proteins, monoclonal antibodies, peptides, molecules that recognize cell surface receptors such as steroid, growth factor, cytokine, other molecules involved in the growth, metabolism, and function of the cell, and the like. In one embodiment, the targeting moiety is directed at a biological target such as a cell, a cell receptor, an antigen, or other surface structures or target-specific extra-cellular matrices, such as mucin, collagen, ion channels, and the like. The targeting moiety may be cell-specific. Alternatively, water-soluble polymeric chelator 110 may be directly injected into colloidal or biological structures such as tumors, cells, and the like, where it is immobilized.

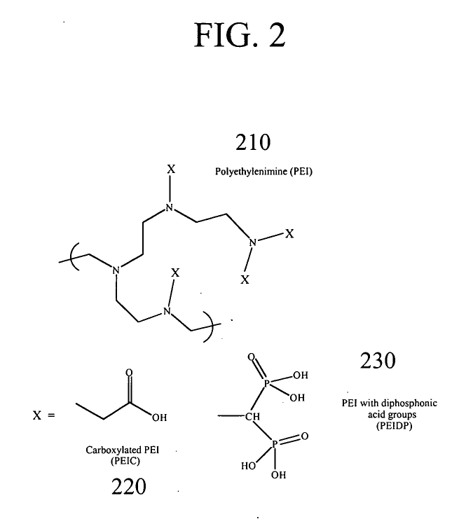

In one embodiment, water-soluble polymeric chelator 110 is a polyethyleneimine (also referred to herein as “PEI”) derivative. Such PEI derivatives include, but not limited to, diphosphonic acid derivatized polyethyleneimines having aminoethylenediphosphonic acid chelating groups, carboxylic acid derivatized polyethyleneimines having aminocarboxylic acid chelating groups, crown chelators, and cryptate chelators. Structures of the cross-linked parent PEI polymer 210, carboxylated PEI 220, and PEI 230 with phosphoric acid groups are shown in FIG. 2.

PEI is a commercially available polymer composed of monomeric aziridine units and has variety of uses in the food packaging and biological stabilization/separations industries. Various sizes of the base polymer PEI have been used as ‘carriers’ for intracellular delivery of viruses, DNA, and other nucleotides.

In contrast to rigid dendrimers and linear polypeptides, PEI has a labile, flexible, three-dimensional structure that allows the molecule to move around, increasing the potential for “catching” and binding daughter nuclei.

PEI is commonly available as hyper-branched polymers that are soluble in aqueous solutions and have a molecular weight (MW) in a range from about 30,000 to about 100,000. The high molecular weights of metallated PEI polymers facilitate their preparation and purification; uncomplexed metals can be easily separated from complexed metals by ultrafiltration. PEI has any active sites; water-soluble polymeric chelator 110 typically has from about 100 to about 700 such chelating groups/active sites. A 50,000 MW polymer PEI globule, for example, contains at least 100 amine sites with primary amines, secondary amines, and tertiary amines are present in a ratio of about 1:2:1. Partial derivatization may be used to create metal-binding sites while still leaving sites available for recognition agent attachment and/or further modification of physical and chemical properties. The excess of active binding sites allows for facile adjustment of the isotope loading level per delivery system. The high concentration of amine sites makes the base polymer cationic within the biological pH range of 5-8.5. With proper tuning, however, PEI derivatives can be zwitterionic or even anionic at the biological pH.

The plurality of radionuclide atoms 120 may include at least one of high energy linear energy transfer radionuclides (such as alpha-emitting radionuclides), low linear energy transfer radionuclides (such as beta-emitting radionuclides), reactor-produced radionuclides such as Re-186, Dy-165, Dy-166, and Lu-177 for therapy purposes, and contrast agents such as iodine, gadolinium, and manganese. These radionuclides may-include the ‘parent’ radionuclide that is bound to water-soluble polymeric chelator 110 for therapeutic purposes, as well as any ‘daughter’ radionuclides resulting from the decay of the parent.

Alpha particles have Relative Biological Effectiveness values that are 3-10 times higher than those for beta particles. In general, alpha particles are emitted from a radionuclide with energy of 5-8 MeV. Their large mass (4 amu) and high energy impart considerable momentum to each particle. When injected into a biological organism, that energy is in turn deposited into surrounding tissue, causing significant cell damage within a very limited area. Thus, heavy element alpha-emitters show promise for the treatment of disseminated diseased tissue since they combine high cytotoxicity with an irradiation range of only a few cells.

Some potential candidates may be bound by water-soluble polymeric chelator 110 in this approach include Ac-225, Ra-223, Ra-225, Ra-224, Dy-166, Re-186, and Lu-177. In the case of alpha-emitters, the limited availability of many of the radionuclides suitable for use in therapy and the relatively short half-lives of such alpha-emitters present problems. In some cases, such as Pb-212, Bi-212 and Bi-213, the therapeutic radionuclide is not itself an alpha emitter of consequence, but is instead a conveniently long-lived precursor to an extremely short-lived alpha-emitting daughter. In other cases, such as At-211, Ra-223, Ra-224, and Ac-225, the alpha-emitting parents are sufficiently long-lived for therapeutic applications. Several decay chains, namely Ra-223, Ra-224, Ra-225 and Ac-225, have multiple alpha emissions, and thus offer the promise of increased cytotoxicity if the daughter nuclei can be retained in close proximity to the target cell.

Daughter nuclei of alpha emitters have a recoil energy of about 100 keV. This energy is sufficient to break all chemical bonds to the nuclei and cause substantial damage to the chelate construct. Although the linear penetration range for recoil nuclei is relatively short (<100 nm), half-lives of even a few seconds could allow for significant migration away from the target sites. In some radionuclide systems, such as Ac-225, ancillary toxicity is a serious issue. It is equally clear that dosimetric analysis and predictive capability is dangerously complicated by the uncertainty related to daughter isotope migration.

Water-soluble polymeric chelator 110 is extremely hydrophilic, forming a diffuse sphere in water. An average 50,000 MW unit of hydrated PEI, for example, is about 95% water and has a solution diameter of about 90 nm. Calculations show that the penetration distance for Fr-221, the first daughter of Ac-225, is 60-105 nm with a dispersion ‘cone’ of about 12 nm in water. Thus, PEI polymers have the potential to retain some fraction of daughter radionuclides, thereby increasing the effective yield at the target. Retention of the daughters within the perimeter of the polymer matrix is aided by ionic interactions and re-complexation with chemical functionalities. Because of the high degree of crosslinking, PEI and its derivatives are resistant to radiolytic degradation, and the large number of binding sites within the polymer favor more complete sequestering of metal ions from the contact solution while decreasing metal exchange rates with biological cations. While the PEI derivatives described herein are not particularly selective, metallation of the polymers is both extremely rapid and highly effective in dilute solutions.

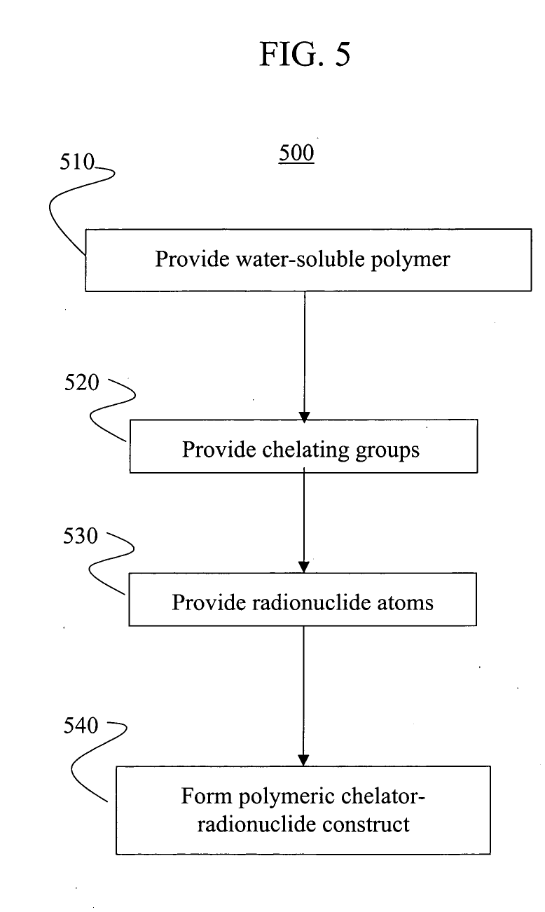

The invention also provides a method of making the radionuclide delivery system described hereinabove. A flow chart of the method 500 is shown in FIG. 5. In Step 510, a water-soluble polymer is provided. Chelating groups and radionuclide atoms are provided in Steps 520 and 530, respectively. In Step 540, a water-soluble polymeric-chelator-radionuclide construct, in which a portion of the radionuclide atoms bind to the chelators that are linked to the water-soluble polymer, is formed. Chelators, radionuclide atoms, and water-soluble polymers are brought into contact with each other in aqueous solution. In one embodiment, the chelates are first linked to the water-soluble polymer, and the radionuclide atoms are then bound to the chelators. Alternatively, the radionuclide atoms first bind to the chelates, and the chelates are then linked to the water-soluble polymer. It is understood that a single radionuclide atom binds to a single chelate and that not all of the chelates bind to radionuclide atoms. Additional chelates or targeting moieties may bind to any vacant sites.

In another embodiment, the method further includes the step of binding a targeting moiety to the water-soluble polymeric-chelator radionuclide construct. The sequence in which the targeting moiety, chelators, and radionuclide atoms are bound to the water-soluble polymer may vary. In one embodiment, the targeting molecule is first bound to the water-soluble polymer, the chelator is then linked to the water-soluble polymer, followed by reaction to bind radionuclide atoms to the chelators, and subsequently filling remaining sites with additional chelates. Alternatively, binding of the targeting moiety is first followed by binding the radionuclide atoms to the chelates to form a complex or complexes, binding the complex (or complexes) to the water-soluble polymer, and finally filling remaining sites with chelates. For PEI, more efficient binding of the radionuclide atoms appears to result from pre-chelation of the polymer (90+% binding) rather than from formation of the chelate-metal complex followed by binding to the polymer (about 50% binding).

Method 500 may further include purification of the water-soluble polymeric-chelator radionuclide construct by at least one of ultrafiltration, electrophoresis, affinity chromatography, dialysis, and combinations thereof.

The following examples serve to illustrate the features and advantages of the present invention, and in no way are intended to limit the invention thereto.

### EXAMPLE 1

### Polymers

Two PEI derivatives, polyethyleneimine (PEI) derivatized with diphosphonic acids (PEIDP) and PEI derivatized with carboxylic acids (PEIC) were evaluated. Schematic representations of the structures of the cross-linked parent PEI polymer 210, carboxylated PEI 220 and PEI with phosphoric acid groups 230 are shown in FIG. 2. In the preparation of both PEIDP and PEIC, water-free PEI was prepurified to >30,000 MW using A/G Technology hollow fiber filters. PEI was then functionalized with carboxylate groups using four equivalents of bromoacetic acid to yield fully-functionalized PEIC. While no detailed structure of the polymer has been obtained, it is believed that both primary and secondary amines are modified to produce aminocarboxylate binding sites similar to those present in EDTA. For a nominal 50,000 MW polymer, functionalization would correspond to approximately 800 aminocarboxylate units per polymeric unit.

The diphosphonic acid derivative (PEIDP) was synthesized in a manner analogous to that of the phosphonic acid derivative by the reaction of formaldehyde and phosphorous with the PEI, as described by B. F. Smith et al. in the Journal of Radioanalytical and Nuclear Chemistry, volume 234, nos. 1-2 (1998) pp. 219-223, the contents of which are incorporated herein by reference in their entirety. Although the polymer structure is not determined, it is likely that at least one diphosphonic acid group is attached to each primary and secondary amine, with the possibility of two groups being bound to each primary amine. The hydrated polymer structure is sufficiently flexible to allow chelating groups on adjacent and non-adjacent sites to contribute to the chemical binding of each metal.

Prior to use in contact experiments, aqueous solutions of each polymer were sized to molecular weights (MW) between 30,000 and 100,000 using ultrafiltration. These solutions were lyophilized, and the solids were reconstituted in acetate buffer to provide stock solutions of 1% polymer by weight (10 mg/mL).

### EXAMPLE 2

### Radionuclide Solutions and Assay

Unless otherwise noted, Am-241 (alpha-emitter, 432 year half-life) and Eu-152 (decay by electron capture (ec), 13.6 year half-life) were used as radiochemical surrogates for actinium for the initial studies, due in part to the short half-life of Ac-225. The few published binding constants for Ac (III) and its behavior on ion-exchange media indicate that actinium's chemical behavior is bracketed by the trivalent lanthanides and actinides. Since the half-life of Ac-225 (alpha-emitter, 10 day half-life) is much shorter than those of the two surrogates, the standard activity loading using the surrogates corresponds to an actinium activity loading of >500 times greater. While the studies reported here focused on actinium and its surrogates, the opportunity to examine the behavior of isolated Bi-213 was exploited as well.

Stock solutions of Am-241 and Eu-152 in 1 molar nitric acid were on the order of 10⁷ dpm/mL. The most precise and consistent assay of stock and contact solutions was afforded by liquid scintillation counting. Assays using gamma counting for these isotopes yielded similar, but slightly more variable, values. While the Am-241 was nearly isotopically pure, the Eu-152 contained significant concentrations of non-radioactive Eu and Gd. Thus, even at tracer concentrations of Eu-152, the polymer sites were nearly saturated with competing trivalent metals.

Ac-225 and Bi-213 were acquired from a ‘spent’ bismuth generator column of <0.3 mCi. Bismuth was eluted from the column using 0.1M HI, yielding a stock solution having an alpha activity of approximately 4×10⁷ dpm/mL. The Ac-225 was recovered from the column using a solution of 0.001M EDTA in pH 4.7 0.1M sodium acetate buffer to strip the actinium from the column. The effluent was concentrated to dryness by heating to a temperature in a range from 100° C. to 150° C. The organics were destroyed by using 10 drops of concentrated nitric acid and 4 drops of 30% hydrogen peroxide and heating to dryness. The residue was reconstituted using 0.5 mL of 0.12 M nitric acid to yield a stock having a of total alpha activity 3×10⁸ dpm/mL.

An assay of both Bi-213 and Ac-225 was conducted on a gamma counter using decay-corrected data and a standard configuration of 5.0 mL of solution in polyethylene scintillation vials. The assay of Bi-213 is facilitated by its strong, isolated peak at about 440 keV. Direct assay of Ac-225 is complicated by its multitude of relatively weak peaks; the most satisfactory method was to wait a day before beginning assay to allow both the Fr-221 (218 keV) and Bi-213 daughters to re-achieve secular equilibrium with parent Ac-225. The francium and bismuth peaks were thus used to determine actinium activities, and the Ac-225 peaks at 100 keV and 150 keV were used only to cross-check the calculated values. At least two decay-corrected assay cycles were conducted for each set of contact experiments. ‘Initial’ and ‘final’ samples were assayed consecutively in order to minimize the effects of decay-correction.

### EXAMPLE 3

### Binding Radionuclides to Polymers

5 mL solutions of buffered saline containing 0.01-0.02% polymer by weight are spiked to approximately 105 dpm/mL (0.05 μCi/mL) using 20-50 μL aliquots of the radionuclide stocks. Solutions are mixed in polyethylene scintillation vials and allowed to sit for varying amounts of time, ranging from 5 minutes to several days, prior to separation. Separation of uncomplexed metals from polymer-bound metals is affected by centrifugation of aliquots to approximately 20-50% original volume through Amicon Centricon® or Amicon Ultra-4® 10 K ultrafiltration units.

### EXAMPLE 4

### Contact Studies

Chemical binding studies using surrogates were carried out in pH 4.2 and pH 4.7 acetate buffered saline (ABS, 0.05-0.1 M acetate, 9 g/L NaCl). For studies using Am-241 and Eu-152, 5 mL solutions containing 0.01-0.02% polymer by weight and ‘blank’ solutions without polymers were spiked to approximately 10⁵ dpm/mL (0.05 μCi/mL) using 20-50 μL aliquots of the radionuclide stocks. Solutions were mixed in polyethylene scintillation vials and allowed to sit for varying amounts of time, ranging from 5 minutes to several days, prior to separation. Separation of uncomplexed metals from polymer-bound metals was affected by centrifugation of aliquots to approximately 20-50% original volume through Amicon Centricon® or Amicon Ultra-4® 10 K ultrafiltration units. The permeates were transferred into tared polyethylene scintillation vials containing approximately 19 mL of scintillant (Ultima Gold® A/B, Perkin Elmer), and an accurate mass of each solution was determined by weighing. The activity of each permeate solution was counted and compared to the activity of the corresponding initial solution (0.1 mL in 20 mL Ultima Gold® A/B) to determine the percentage of isotope retained. A typical run mixed 4.9 mL of buffer solution, 50-100 μL of PEIC or PEIDP 1% stock, and 20-50 μl of radionuclide stock. Each 5 mL of solution afforded two 2 mL aliquots as duplicates for ultrafiltration separation.

Chemical binding studies in which Am-241 bound to PEIDP was challenged with biological cations were conducted in pH 4.7 0.05M acetate buffer. In this procedure, an excess of the biological cation is added to the radionuclide/polymer construct to see if the biological cations are capable of displacing the radionuclide. Aliquots of a concentrated stock of NaCl (68 g/L) were spiked into buffered solutions of the metal/polymer complex to provide 0 g/L NaCl, 6.2 g/L NaCl, and 11 g/L NaCl solutions. Physical separations and assays were performed as outlined above. Further studies added aliquots of aqueous stock solutions of calcium chloride (0.472 M Ca⁺²) or ferric nitrate (2 M Fe⁺³) to solutions of Am-241 with PEIDP in pH 4.7 ABS.

In repeated-contact studies, samples were reduced to 15-25% of their original volume using centrifugation through a 10,000 MW cutoff filter. The permeate was assayed and its activity subtracted from the initial sample to determine the activity remaining in the retenate. The retentate, which contained polymer-bound metal, was then reconstituted to its original volume. This solution was mixed and concentrated again. Assay of the radionuclide activity of the second permeate was compared to the calculated activity of its parent solution. A third cycle was performed, again using a permeate assay to re-calculate the activity remaining bound to the polymer. These studies were of a scoping nature only, since the uncertainties associated with measurement rapidly accumulate.

For studies using Bi-213, the initial activity of the samples was increased to about 5×10⁵ dpm/mL to compensate for the activity decay during contact, separation, and transport for gamma counting. Aliquots of 100 μL stock were used in 10 mL total volume with either 0.01 weight % polymer, or ‘blank’ solutions without polymer, in the two ABS solutions. After aliquots of 0.1 mL were removed for ‘initial’ solution activity measurements, the remainder of each sample was centrifuged to approximately 20-40% volume through Amicon Ultra-15® 10 K ultrafiltration units (10 minutes). 5.0 mL aliquots of these ‘final’ solutions were pipetted into vials for gamma assay. Two separate sets of elution and contact studies were performed using Bi-213 prior to stripping the column of Ac-225. Studies using Ac-225 were conducted similarly to the Bi-213 studies, except for using 20 μL aliquots of the stock solution.

### EXAMPLE 5

### Protein Conjugation

Each polymer was ‘tagged’ with green fluorophore 8-aminopyrene-1,3,6-trisulfonic acid (APTS) to facilitate visual tracking of the protein conjugation reaction and to provide a fluorescent handle for flow cytometry experiments. The carboxylate and diphosphonate groups of the polymers were activated using 1-ethyl-3-(3-dimethylaminopropyl) carbodiimide, hydrochloride (EDAC) in water with the pH adjusted to 5.8-6.0. Aliquots of a stock solution of APTS in water (1 g/mL) were added to the polymer/EDAC solutions at a ratio of 0.5 to 1 mole APTS to 100 chelate groups, giving a tagging label from 0.5% to 1%. Ratios are not precise, since the actual composition of the polymer is not well defined. The mixtures were stirred for 30 minutes and then transferred to 10,000 MW ultrafiltration tubes. All of the green fluorophore was retained with the polymer, indicating quantitative reaction. Polymers were washed three times with deionized water to remove excess EDC, and allowed to sit in water overnight to hydrolyze unreacted sites back to the original chelate. Solutions were lyophilized to a bright green solid. Supplies of tagged PEIDP and PEIC polymers were made by combining 50 mg of polymer (about 0.4 mmoles of active sites) with 7.9 mg of EDAC (0.04 mmoles) and 0.004 moles of APTS.

Conjugation of lectin protein Concanavalin A (ConA, Sigma, type VI) to each tagged polymer used a similar method to couple lysines of the Con A to the carboxylate and diphosphonate groups of the polymer. The ConA was reconstituted to a stock solution of 5 mg/mL in HEPES buffered saline (HBS; pH 7.2, 0.25 molar HEPES, 1 mM calcium chloride, 0.1 mM manganese chloride, 0.15M sodium chloride). At this pH, ConA exists as a tetramer having a molecular weight of 104,000, which was approximately twice the MW of the polymers. Successful conjugation of the two large molecules required protection of the ConA active site with methyl-a-D-mannopyranoside and concentration of the reaction solutions to about 10-20 mg/mL. In a typical reaction, 5 mg of ConA, 1-2 mg of methyl-D-monoxide, and 1 mg of APTS tagged PEIDP or PEIC were combined in the HBS and concentrated using centrifugation in an Amicon Ultra-15® 10,000 MW unit to approximately 0.25 mL total volume. After adding 2 mg of EDAC (10 mg/mL in HBS), the solution was re-concentrated to about 0.25-0.5 mL and allowed to stand for several hours (3 hours at room temperature, or overnight at 3° C.). Phosphate-buffered saline was then added to react with excess EDAC-activated sites, and about 2 mg of methyl-a-D-glucopyranoside in HBS was added to displace the mannose from the ConA active sites. The reaction solution was diluted to 15 mL with HBS and concentrated to 0.5 mL in the Amicon Ultra-15® tubes through three cycles to remove excess EDAC, glucose and mannose. Separation of the ConA conjugated, APTS-tagged polymers from unconjugated polymers was achieved by running the concentrated solution down a column of D-mannose on agarose (Sigma) in HBS. Unconjugated polymer was not retained on the affinity column, and was eluted in HBS as a green band. Conjugated polymer was retained as a green band that was eluted with a solution of methyl-a-D-mannopyranoside (1 mg/mL in HBS). This method does not separate free ConA from polymer-conjugated ConA.

Eluants were washed in an Amicon Ultra-15® 100 MW unit three times as above using phosphate buffered saline (PBS, Sigma, pH 7.2) to remove excess HEPES, mannose, and possibly unconjugated ConA. Only trace amounts of the conjugated systems (ConA/tagged-PEIC or ConA/tagged-PEIDP) were found in the permeate from these washings. Samples were diluted to a total volume of 1 mL for assay.

Assay of the ratio of ConA to polymer in the eluant was conducted using UV and visible absorbances in PBS. Calibration curves were established for the ConA at 279 nm using four solutions ranging from 0.0625 mg/mL to 0.5 mg/mL, and for the APTS-tagged PEIC and APTS-tagged PEIDP at 299 nm and 442 nm, repectively, using four solutions ranging from 0.027 mg/mL to 0.21 mg/mL. Ratios of ConA to APTS-tagged polymer were assayed to be 2 mg/mL ConA to 0.27 mg/mL PEIC, and 0.82 mg/mL ConA to 0.26 mg/mL PEIDP.

### EXAMPLE 6

### Metal/Polymer Interactions

As previously mentioned, Am-241 and Eu-152 were used as radiochemical surrogates in initial studies, were used as radiochemical surrogates for actinium for the initial studies, due in part to the short half-life of Ac-225. Also, although the project focused on actinium and its surrogates, the behavior of isolated Bi-213 as was studied as well.

Centrifugation-based ultrafiltration is an effective method to quickly separate free metal from polymer-bound metal. Amicon Ultra® tubes have a much larger surface area to volume ratio than the corresponding Amicon Centricon® tubes, and afford a more rapid separation (e.g., 10 minutes vs. 60 minutes). Ultrafiltration, however, cannot distinguish between polymer-bound metal and other metal colloids or precipitates larger than 10,000 MW. Since trivalent actinides and lanthanides hydrolyze at relatively low pH, this study was restricted to solutions well below the physiological pH range of 7.2-7.4 in order to unequivocally differentiate between metals bound to the polymeric chelators and metals that are present as part of other macromolecular species or attached to container surfaces.

Hydrolysis of trivalent metals in neutral solutions can significantly affect their ability to complex quickly and quantitatively to the desired chelator. The ionic radii (trivalent, 6-coordinate) of the Eu and Am surrogates are slightly smaller than for Bi and Ac (95 pm and 98 pm vs. 103 pm and 112 pm), it is therefore expected that the surrogates would reflect a worst-case scenario regarding hydrolysis. Indeed, recent studies have shown that tracer concentrations of Ac(III) exhibit the expected trend of being slightly less susceptible to hydrolysis than its homologue, La(III). Trivalent bismuth, however, exhibits an unduly high tendency to hydrolyze and form ionic and neutral complexes that have the potential to interfere with its ability to bind to chelators at relatively low pH regimes.

“Blank” solutions of Am, Eu, Bi and Ac with no-polymer-added display little (<11%) retention of metal when using acetate buffered saline at pH 4.2 (ABS 4.2) and pH 4.7 (ABS 4.7). MES (2-(4-morpholino)ethanesulfonic Acid)-buffered salines at pH 5.1 and 5.6, however, display unacceptably high retention of metal (>95%, as determined using Eu-152) indicating that significant hydrolysis and/or competing reactions occur in these higher pH, non-complexing solutions. Thus, these buffer solutions were considered to be unsuitable for further study. Even in the ABS solutions, where formation of macromolecular complexes is low, the formation of small charged and neutral complexes could still adversely affect metal-binding to the polymer chelating sites.

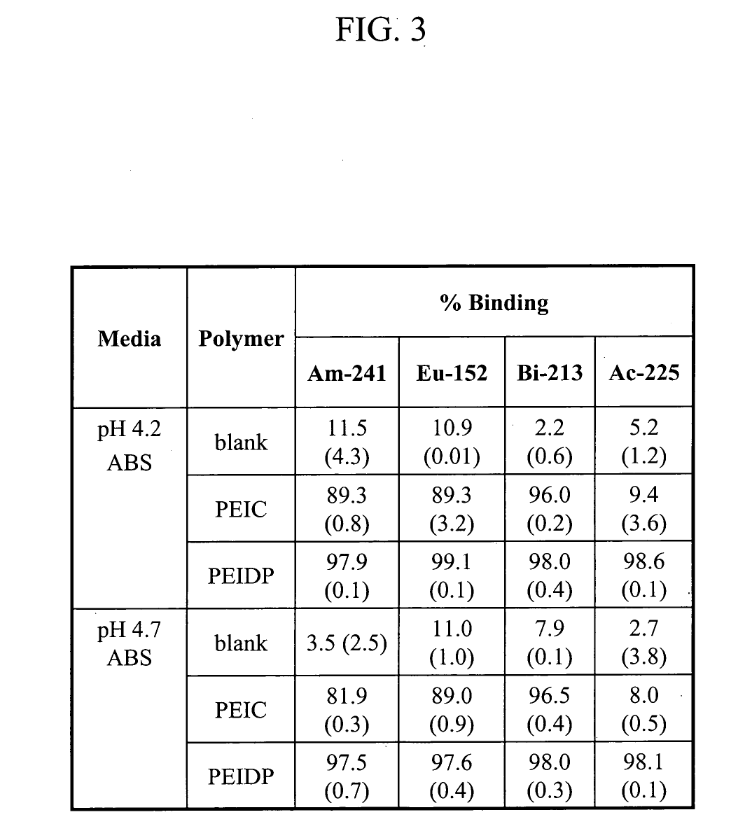

The metal-polymer binding data for the radionuclides, media, and polymers studied are shown in FIG. 3. The data are not time-resolved, as it was found that the kinetics of metal sorption are fast relative to the amount of time required for centrifugation (>10 minutes). The percent uptake data presented in the table and their corresponding uncertainty levels are based on a minimum of two sets of duplicate experiments. The aminocarboxylates of PEIC—particularly those derived from the primary amines of the parent PEI—may be reasonably compared to EDTA, imidodiacetic acid (IDA), and ethylenediiminodiacetic acid (EDDA) where the dissociation constants (pKa's) of the first two carboxylates are on the order of 2 to 3. There are fewer published analogues of the active sites of PEIDP, but the first pKa of amino- and methylamino-methylenediphosphonic acid is approximately 1.4. Thus, for both PEIC and PEIDP, the chelating sites should be available for metal binding under the experimental conditions using the two acetic acid/acetate buffers. Indeed, little difference in the metal-binding characteristics between the two sets of buffers of about 0.5 pH units separation is actually observed.

Both PEIC and PEIDP exhibit consistent behavior and good affinities for Am-241 and Eu-152. PEIC is less competitive in the acetate buffers than PEIDP, with about 80-90% metal uptake in PEIC versus about 97-99% metal uptake in PEIDP. Metal complexes of the PEIC polymers exhibit a small, but reproducible shift in which the binding increases slightly during the first few hours (89% to 90%) and then decreases over a period of several weeks to as low as 86%. Metal binding to the PEIDP polymers is much higher and more stable.

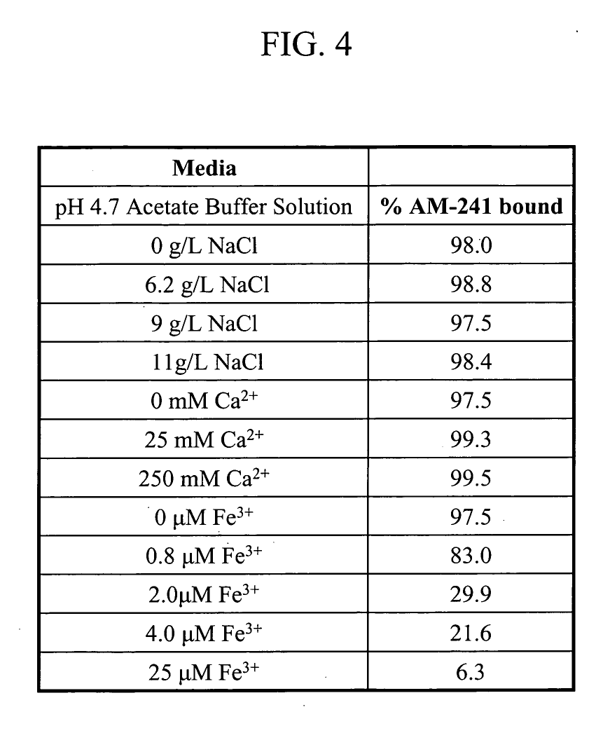

Using various serum formulations as a rough guide to concentrations of monovalent (Na/K), divalent (Ca/Mg) and trivalent (Fe) biological cations, experiments in which the Am-241/PEIDP construct was challenged with Na, Ca, and Fe ions. The results of these studies, which are summarized in the table shown in FIG. 4, establish that the bound metal is not displaced by any of these metals at substantial excess to biological conditions. In the case of Ca(II), biological concentrations of about 2.6 mM provide an approximately 3-fold excess of Ca(II) to PEIDP binding sites under experimental contact conditions. Quantities of calcium in 100-fold excess of this value have no impact upon the ability of the polymer to retain Am(III). As a trivalent species, free iron is a stronger competitor, but biological concentrations of free iron are extremely low (approximately 0.25 μM). Experimental contact solutions contain about 800 μM of chelating polymer sites. However, Fe(III) does not begin to displace Am(III) until this concentration of free iron is reached; i.e., until the polymer sites have become saturated with iron at about 3000 times the biological concentration of Fe(III). At small excesses to saturation (0.8 μM), approximately 17% of the Am(III) is displaced. Displacement appears to level off at 80% by the time a 6-fold excess of the biological FE(III) concentration is reached. Based on these results, it is clear that the active sites of PEIDP are not selective for Am(III) (nor presumably for Ac(III)), but this lack of selectivity may be of little consequence under biological conditions.

Whereas binding of Am-241 and Eu-152 to the PEIDP polymer is uniformly high, preliminary repeated-contact studies show that binding to the PEIC polymer can be enhanced by using a 10,000 MW cutoff filter to remove uncomplexed metal or small metal/polymer complexes from the initial contact solution. In a pH 4.2 acetate buffered solution (ABS 4.2), the percentage of Am-241 that is bound to the polymeric chelator rises from 89% to 94% with a single ‘wash.’ In a pH 4.7 acetate buffered solution (ABS 4.7), the percentage of Eu-152 bound to the polymeric chelator rises up to 98% for two such washes. This observed increase in metal-binding of the PEIC polymer may indicate that some ‘fixed’ portion of the radionuclide is unavailable for complexation, that small units of the PEIC were not removed during the initial ‘sizing’ of the polymers, or, in the case of Eu-152, that “stable” (i.e., not radioactive) trivalent metal saturates the active sites. Thus, the actual equilibrium constants for binding trivalent metals with the aminocarboxylate sites of PEIC may be higher than is indicated by the relatively modest 80-90% observed with the single contact. The true extent of the enhancement in metal binding is uncertain, since the errors associated with each contact/activity calculation accumulate rapidly.

Bi-213 exhibits high (98%) levels of binding to PEIDP, and binding to the PEIC is higher (96%) than observed for Am or Eu. In general, complexation chemistry of bismuth is similar to ‘hard’ trivalents and tetravalents. For example, stability constants for ML (multi-ligand) complexes of EDTA with Bi(III), Th(IV), and Fe(III) are up to 8 orders of magnitude greater than those of Am(III), Pu(III), and Eu(III). Calibrated gamma counts indicate that the Bi-213 concentrations in the contact solutions were about 10-13 M. Higher stability constants and lower bismuth concentration clearly favor more comprehensive sequestration. Given the tendency of Bi(III) to hydrolyze, the ‘blank’ solutions indicate low formation of macromolecular species in the two ABS solutions. The low retention of Bi in ABS indicates that the acetate complexes with bismuth, forming a discrete metal complex that remains soluble in water (i.e., is present in the permeate). If the acetate had not bound to the Bi, a Bi(OH)ₓ colloid would form, and that colloid would be retained by the filter in the retenate (as is observed for non-complexing MES) at a similar pH.

Although actinium binds well (98%) to PEIDP, unexpectedly low Ac-225 binding to the PEIC polymer was observed. The observed interactions of Ac-225 with PEIC are inconsistent with results obtained for the Eu-152 surrogate and Bi-213. One possible explanation for this behavior is that small quantities of a competing complexant were present in the actinium solution. The complexant may have originated from degradation of the ion-exchange material or from leaching of glassware with the concentrated nitric acid/hydrogen peroxide solution that is used to destroy degradation products. Whatever the reasons for the poor PEIC interactions, the high level of binding of Ac-225 to the PEIDP polymer is clearly in line with the expected chemistry. Calibrated gamma counts indicate that the contact solutions had an Ac-225 concentration in the 10-11 M range.

An attempt was made to determine the extent of primary daughter retention by the polymers using Ac-225, but the short half-life (5 minutes) of the Fr-221 daughter makes it difficult to measure Ac-225 retention directly under diffusion-controlled conditions. In addition, the high affinity of the polymers for the Bi-213 daughter means that neither this daughter nor the longer lived (3 hour) Pb-209 daughter cannot be used to back-calculate free Fr-221 concentrations. Gentle centrifugation through the ultrafiltration units is much too slow relative to the Fr-221 half-life, and vigorous centrifugation would be expected to expel all metals that are not strongly chemically bound to the polymer (Fr(I) would not be strongly bound to the polymer). However, contact experiments bringing the Bi-213 daughter into secular equilibrium with its Ac-225 parent show that this daughter nucleus is strongly bound to the polymers—either completely through re-uptake by polymer sites or through a combination of physical retention and re-uptake.

### EXAMPLE 7

### Polymer/Biomolecule Conjugation

The effect of conjugating the polymer to a biologically active molecule was tested by utilizing Concanavalin A (ConA), a plant lectin that binds to mannose residues common to most cell types. At neutral pH, ConA exists as a tetramer having a MW of 104,000, or approximately twice the MW of the polymers, but is physically much more compact. The stoichiometry of the conjugation of ConA with the polymers could not be well controlled due to the multiplicity of amines on the protein and the carboxylates or phosphonates on the polymers. Attempts to control stoichiometry by activating only a small fraction of the polymer sites with EDAC failed to produce any conjugate. Only when we saturated the polymer carboxylate or phosphonate sites with EDAC and contacted the fully-activated system with the ConA under fairly concentrated conditions did we obtain low yields of the protein/polymer conjugate. In addition, it also appeared that the sugar-binding site of the ConA needs to be protected during the one-pot conjugation using excess EDAC. At least, in the absence of mannose or glucose, no ConA/polymer conjugate was isolated on the mannose affinity column. Other researchers, however, have conducted similar one-pot reactions with ConA and EDAC, and have not reported the need for binding-site protection.

As evidenced by a visibly green eluant associated with the HBS front, substantial amounts of unreacted APTS-polymers are present in the reaction mixture, which is indicative of low efficiency for the protein conjugation. No ConA was detected in the HBS eluant. The retained green band, however, substantiates that some fraction of the reactants coupled with the HBS, and that coupling of the ConA to the large, diffuse polymer does not significantly inhibit its ability to bind to mannose. An assay of this band, eluted with mannose, reveals high ratios of ConA to polymer, particularly for the PEIC system. Simple washing of the mannose eluant though an Amicon Ultra-15® 100,000 MW unit removed some unreacted ConA, but no further purification was deemed necessary for this straightforward proof-of-principle study.

The polymeric chelators studied display a high affinity for radioisotopes of interest, such as Ac-225 and Bi-213. While the chelating moieties, aminomethylenediphosphonates (PEIDP) and aminocarboxylates (PEIC), are not highly selective for these particular isotopes, these studies show that PEIDP and PEIC may be sufficiently selective for trivalent metals to afford a level of biological stability that is satisfactory for therapeutic applications.

While typical embodiments have been set forth for the purpose of illustration, the foregoing description should not be deemed to be a limitation on the scope of the invention. Accordingly, various modifications, adaptations, and alternatives may occur to one skilled in the art without departing from the spirit and scope of the present invention.

1. A radionuclide delivery system, the delivery system comprising:
a. a water-soluble polymeric chelator having a plurality of chelating groups; and
b. a plurality of radionuclide atoms, wherein each of the radionuclide atoms is bound to one of the chelating groups, wherein the plurality of chelating groups immobilize at least a portion of the plurality of radionuclide atoms.

2. The delivery system according to claim 1, further comprising a targeting moiety bound to the water-soluble polymeric chelator.

3. The delivery system according to claim 2, wherein the targeting moiety is selected from the group consisting of proteins, monoclonal antibodies, peptides, and molecules that recognize cell surface receptors, wherein the cell receptors comprise at least one of steroids, growth factors, cytokines, and molecules involved in cell growth, cell metabolism, and cell function.

4. The delivery system according claim 2, wherein the targeting moiety is directed to a biological target.

5. The delivery system according to claim 4, wherein the biological target is one of a cell, a cell receptor, an antigen, a target-specific extra-cellular matrix, an ion channel, mucin, and collagen.

6. The delivery system according to claim 4, wherein the targeting moiety is cell specific.

7. The delivery system according to claim 1, wherein the water-soluble polymeric chelator is a polyethyleneimine derivative.

8. The delivery system according to claim 7, wherein the polyethyleneimine derivative is one of a diphosphonic acid derivatized polyethyleneimine and a carboxylic acid derivatized polyethyleneimine.

9. The delivery system according to claim 8, wherein the diphosphonic acid derivatized polyethyleneimine includes a plurality of aminoethylenediphosphonic acid chelating groups.

10. The delivery system according to claim 8, wherein the carboxylic acid derivatized polyethyleneimine includes a plurality of aminocarboxylic acid chelating groups.

11. The delivery system according to claim 1, wherein the water-soluble polymeric chelator has a molecular weight in a range from about 30,000 to about 100,000.

12. The delivery system according to claim 1, wherein the water-soluble polymeric chelator has at least 100 chelating groups.

13. The delivery system according to claim 1, wherein the plurality of radionuclide atoms comprises at least one of high linear energy transfer radionuclides, low energy linear energy transfer radionuclides, reactor-produced radionuclides, contrast agents, and daughters thereof.

14. The delivery system according to claim 13, wherein the plurality of radionuclide atoms comprises at least one of alpha emitters, beta emitters, and gamma emitters.

15. The delivery system according to claim 14, wherein at least a portion of the radionuclide atoms are alpha emitters.

16. The delivery system according to claim 14, wherein the plurality of radionuclide atoms includes radionuclide atoms and daughter nuclei thereof.

17. The delivery system according to claim 16, wherein the daughter nuclei have a recoil energy of about 100 eV.

18. The delivery system according to claim 16, wherein the plurality of radionuclides comprises radionuclides selected from the group consisting of Ac-225, Ra-223, Ra-225, Ra-224, Dy-166, Re-186, At-211, Pb-212, Bi-212, Bi-213 and Lu-177.

19. A water-soluble polymeric chelator, wherein the water-soluble polymeric chelator is a polyethyleneimine derivative having a plurality of chelating groups, wherein each of the chelating groups is capable of binding and immobilizing a radionuclide atom.

20. The water-soluble polymeric chelator according to claim 19, further comprising a target moiety bound to the water-soluble polymer.

21. The water-soluble polymeric chelator according to claim 19, wherein the targeting moiety is selected from the group consisting of proteins, monoclonal antibodies, peptides, and molecules that recognize cell surface receptors, wherein the cell receptors comprise at least one of steroids, growth factors, cytokines, and molecules involved in cell growth, cell metabolism, and cell function.

22. The water-soluble polymeric chelator according claim 19, wherein the targeting moiety is directed to a biological target.

23. The water-soluble polymeric chelator according to claim 22, wherein the biological target is one of a cell, a cell receptor, an antigen, a target-specific extra-cellular matrix, ion channels, mucin, and collagen.

24. The water-soluble polymeric chelator according to claim 22, wherein the targeting moiety is cell specific.

25. The water-soluble polymeric chelator according to claim 19, wherein the polyethyleneimine derivative is one of a diphosphonic acid derivatized polyethyleneimine and a carboxylic acid derivatized polyethyleneimine.

26. The water-soluble polymeric chelator according to claim 25, wherein the diphosphonic acid derivatized polyethyleneimine includes a plurality of aminoethylenediphosphonic acid chelating groups.

27. The water-soluble polymeric chelator according to claim 25, wherein the carboxylic acid derivatized polyethyleneimine includes a plurality of aminocarboxylic acid chelating groups.

28. The water-soluble polymeric chelator according to claim 19, wherein the water-soluble polymeric chelator has a molecular weight in a range from about 30,000 to about 100,000.

29. The water-soluble polymeric chelator according to claim 19, wherein the water-soluble polymeric chelator has at least 100 chelating groups.

30. A radionuclide delivery system, the delivery system comprising:
a. a water-soluble polymeric chelator having a plurality of chelating groups, wherein each of the chelating groups is capable of binding and immobilizing a radionuclide atom; and
b. a plurality of radionuclide atoms, wherein each of the radionuclide atoms is bound to one of the chelating groups; and
c. a targeting moiety bound to the water-soluble polymeric chelator.

31. A method of making a radionuclide delivery system, wherein the delivery system comprises a water-soluble polymeric chelator-radionuclide construct comprising a water-soluble polymeric chelator having a plurality of chelating groups, wherein each of the chelating groups is capable of binding and immobilizing a radionuclide atom; and a plurality of radionuclide atoms, wherein each radionuclide atom is bound to one of the chelating groups, the method comprising the steps of:
a. providing the water-soluble polymeric chelator;
b. providing the plurality of chelating groups;
c. providing the plurality of radionuclide atoms; and
d. forming the water-soluble polymeric chelator-radionuclide construct, wherein a portion of the radionuclide atoms bind to a portion of the chelator groups and the chelator groups are linked to the water-soluble polymer.

32. The method according to claim 31, wherein the step of forming the water-soluble polymeric chelator-radionuclide construct comprises:
a. linking the plurality of chelators to the water-soluble polymer to form the water-soluble polymeric chelator; and
b. binding a portion of the plurality of radionuclide atoms to the chelators linked to the water-soluble polymeric chelator.

33. The method according to claim 31, wherein the step of forming the water-soluble polymeric chelator-radionuclide construct comprises:
a. binding a portion of the plurality of radionuclide atoms to the chelators; and
b. linking the plurality of chelators bound to the radionuclide atoms to the water-soluble polymer to form the water-soluble polymeric chelator construct.

34. The method according to claim 31, further comprising the step of attaching a targeting agent to the water-soluble polymeric chelator construct.

35. The method according to claim 31, further comprising the step of purifying the polymeric chelator-radionuclide construct.

36. The method according to claim 31, wherein the step of purifying the polymeric chelator-radionuclide construct comprises purifying the polymeric chelator-radionuclide construct by at least one of ultrafiltration, electrophoresis, affinity chromatography, dialysis, and combinations thereof.
